(19) United States
(12) Patent Application Publication
(10) Pub. No.: US 2019/0038530 A1
(43) Pub. Date: Feb. 7, 2019

(54) COSMETIC COMPOSITIONS FOR SKIN CARE

(71) Applicant: FAREVA S.A., Luxembourg (LU)

(72) Inventors: Laurent PAGNARD, Tain L'Hermitage (FR); Sandrine VENTRE, Alfortville (FR)

(21) Appl. No.: 15/907,371

(22) Filed: Feb. 28, 2018

(30) Foreign Application Priority Data
Aug. 3, 2017 (FR) FR1757501

(51) Int. Cl. A61K 8/26; A61K 8/34; A61K 8/36; A61K 8/02; A61Q 19/00; A61Q 5/00

(52) U.S. Cl. CPC A61K 8/26; A61K 8/345; A61Q 5/00; A61K 8/0229; A61Q 19/00; A61K 8/361

(57) ABSTRACT

A cosmetic composition that can be heat cast and molded, comprising from 10 to 40% by weight of one or more clays, preferably selected from the following families of clays: kaolin, bentonite, laponite, smectite, illites, chlorites, vermiculites, sepiolites, attapulgites as well as their derivatives and their mixtures and combinations, from 10 to 60% by weight of water, from 5 to 70% by weight of one or more humectants and from 3 to 10% by weight of one or more viscosity agents.

### FIELD OF THE INVENTION

The present invention relates to cosmetic compositions, as well as articles comprising these compositions, for the care of the skin and/or the hair, in particular masks for the face or the hair, and which can be applied in a simple, fast and hygienic manner.

### BACKGROUND OF THE INVENTION

There is undoubtedly a large number of cosmetics applicable to the skin for the purpose of cleaning, exfoliating/scrubbing, moisturizing, treating or applying makeup.

These products can be in anhydrous or aqueous form. These are solid products, such as powders, emulsions, for example milks, creams, or even dispersions, in particular masks. These products can be ready for use or, as often in the case of masks, in a form that may require some preparation, for example by first adding water.

The application of these products depends, of course, to a certain extent on their form, their consistency and their utility, but it is usually done conventionally by portioning a certain quantity of product, followed by spreading or distributing it on the skin or even the hair using the hands or, more rarely, an applicator. However, even in case of the supply of a dedicated applicator, it is not uncommon nevertheless for the user to prefer or to have to complete or improve the application by means of his/her hands.

However, this method is a priori not hygienic, can be “messy” and often requires the user to wash his/her hands after the application. This is particularly true for masks for the face, for example, but also for other cosmetics where the intended location of application is, in fact, not on the fingers or the inner side of the hand.

In addition, certain cosmetic compositions, while they fulfill the purpose of particular care for which they are provided, are, however, not much popular with users, for the reasons mentioned in the paragraph above or because their application is tedious or complicated by their presentation or their implementation in several steps, or because their consistency feels unpleasant or are “immobilizing” for too long a period of time.

### SUMMARY OF THE INVENTION

The present invention relates to a cosmetic composition comprising 10 to 40% by weight one or more clays, 10 to 60% by weight of water, 5 to 70% by weight one or more humectants, and 3 to 10% by weight one or more viscosity agents.

An object of the present invention is therefore to provide novel cosmetic compositions having a presentation facilitating application to the face or other parts of the body, preferably also the hair, or even the nails.

### DETAILED DESCRIPTION OF THE INVENTION

In order to solve at least some of the problems mentioned above, the present invention proposes, in a first aspect, a cosmetic care composition, in solid form, that can be heat cast and molded, comprising from 10 to 40% by weight of one or several clays, preferably selected from the following families of clays: kaolin, bentonite, laponite, smectite, illites, chlorites, vermiculites, sepiolites, attapulgites and their derivatives (in particular any form of clay functionalized or converted by chemical reaction); and mixtures and combinations thereof, from 10 to 60% by weight of water, from 5 to 70% by weight of one or more humectants and from 3 to 10% by weight of one or more viscosity agents.

With the galenic methods of cosmetic compositions of the type indicated, the inventors have developed products which, in addition to their benefits in terms of care, have a consistency that is particularly interesting at application temperatures despite the very high contents in clays, which tend precisely to make the formulation complicated and the composition unstable. Indeed, in cosmetic formulas, such as emulsions for example, the introduction of a high percentage of clay can often cause the composition to become unstable. Advantageously, the cosmetic compositions of the present invention combine several strengths that were thought to be incompatible: the formulas are stable despite a high percentage of clay, they are preferably of the purifying cleansing type, they guarantee a fresh sensation on the skin, they are very easy to rinse and do not leave a greasy film on the skin after rinsing.

In the context of the present invention, the term “humectant agent” has the meaning generally given in the cosmetics field. In this case, these are generally compounds or mixtures of compounds that retain water thanks to their hygroscopic power. Among the particularly suitable humectants, there are in particular urea, amino acids and their derivatives (such as histidine, pidolic acid, etc.), organic acids and their salts (such as glycyrhizic acid, hyaluronic acid, calcium gluconate, sodium lactate, etc.), glycerin (also called glycerol) and all types of polyols (such as ethylene glycols, propylene glycol, triethylene glycol, tripropylene glycol, polyethylene glycol (PEG), sorbitol, etc.), natural and synthetic derivatives of sugars and polysaccharides (such as fructose, glucose, isomalt, lactose, maltitol, mannitol, polydextrose, sucrose, xylitol, etc.), hydrolysates of silk, keratin or soy, and their mixtures and combinations. The humectant(s) used is (are) preferably propylene glycol, glycerin or a combination thereof. Particularly advantageous cosmetic compositions according to the invention generally comprise from 3 to 40% by weight of propylene glycol and from 3 to 30% by weight of glycerin.

The viscosity agents may be selected from those generally known in the cosmetics field. Their role is to allow adjusting the viscosity of the cosmetic composition, in this case to increase it. In certain embodiments, the cosmetic compositions according to the invention can therefore comprise, as viscosity agent(s), one or more fatty acids comprising a carbon chain of between 12 and 22 carbon atoms, neutralized with mineral or organic bases to form mineral or organic salts of these fatty acids, such as, sodium stearate, sodium palmitate, etc. or their mixtures and combinations.

The formulas containing these neutralized fatty acids will thus have a solid form at room temperature. Due to their particular composition, the cosmetic compositions of the invention are fluid at high temperature, which facilitates their handling during casting/molding (such as at a temperature between 45 and 80° C.). Alternatively, or in addition, the cosmetic compositions may also comprise from 0.5 to 10% of a dye, a mineral or organic pigment in solid form or in a slurry, an opacifying agent, pearlescent agents, flakes, exfoliating particles, and their mixtures or combinations.

Alternatively, or in addition, the cosmetic compositions according to the invention may also comprise from 0.01 to 5% by weight, preferably from 0.1 to 4% by weight, of one or more suspending agent(s), preferably selected from xanthan gum, guar gum, locust bean gum, sclerotium gum, gellan gum, cellulose derivatives (such as hydroxy ethyl cellulose, hydroxy methyl cellulose), synthetic polymers (such as acrylate copolymer, polyacrylate copolymer, carbomer, etc.), and their mixtures and combinations. These suspending agents increase, if necessary, the stability of the compositions in the longer term or in adverse conditions.

It may be envisaged to add to the cosmetic compositions of the invention, for example, from 0.1 to 7% by weight, preferably from 0.25 to 5% by weight, of one or more cosmetic active ingredients (for example, detoxifying agents, antipollution agents, etc.), emollients, emulsifiers, fragrances, preserving agents, pH adjusting agents, etc.

A second aspect of the invention relates to articles comprising a cosmetic composition according to the invention which has been heat cast and molded. The heat casting and molding of a cosmetic composition can be done by methods well known to those skilled in the art. More details will be given below. Among the particularly preferred items are sticks. In a particularly preferred manner, a stick according to the invention comprises a cosmetic mask composition for the face and/or the hair.

The main advantages of a face (or hair) mask with a high clay content in a stick according to the invention, compared to existing solutions, include that a stick is more hygienic (no direct contact of the hands with the product), it dries quickly because the combination of the stick form and its consistency makes it easy to deposit the right amount of product, this form is also more practical and less messy than the existing products. Therefore, a stick according to the present invention offers the advantage of containing a high level of clay, up to 25%, or even 40% by weight. As already mentioned above, the introduction of a high percentage of clay, such as kaolin, generally poses significant problems from a formulation standpoint and will almost inevitably cause instability. Thus, the present invention allows to combine the several advantages in a single product, having a stable formula and a high percentage of clay, for example kaolin. In addition to these advantages, the masks according to the present invention are purifying, cleansing, offer a fresh sensation (due to an aqueous formula) and are very easy to rinse. No greasy film will be left on the skin after use.

In a third aspect, the invention provides a method for manufacturing a heat cast and molded article comprising hot mixing of 10 to 40% by weight of one or more clays, as previously described, from 10 to 60% by weight of water, 5 to 70% by weight of one or more humectants, 3 to 10% by weight of one or more viscosity agents and possibly other ingredients to form a cosmetic composition according to the invention. During this mixing step, the operating temperature is preferably adjusted to a value between 45 and 75° C., more preferably between 50 and 70° C. After having obtained an intimate and homogeneous mixture of the constituents, the cosmetic composition thus obtained is cast in a mold, for example in the form of a cylinder with circular section or not, such as straight circular cylinders or oval or polygonal section cylinders, for example, rectangular parallelepipeds or not. The shape of the molded composition will of course depend on the intended use. In the case of a stick, the molded cosmetic composition will have a shape and dimensions appropriate to the sliding or pivoting mechanism of the stick. It should be noted that the casting/molding is advantageously carried out directly in a sliding or pivoting stick device. Once molded, the composition is cooled to a solidification temperature of the composition, preferably at a temperature below or at room temperature, particularly at a temperature between 0 and 25° C.

A fourth aspect of the invention relates to the use of a cosmetic composition or an article according to the invention or to a heat cast and molded article obtained by the manufacturing process according to the invention, in particular a cosmetic article in the form of a stick as described herein, and comprises the application of the cosmetic composition to one or more parts of the body, preferably on the skin, the nails and/or the hair, in particular on the face, the neck, torso, arms, hands, legs and/or feet. Preferably, the cosmetic composition is removed after a period of time by rinsing, preferably with water. The time between application and rinsing may vary, but is usually between 1 and 20 minutes.

The useful amounts of cosmetic composition according to the invention are generally from 1.5 to 10 mg/cm², preferably from 3 to 8 mg/cm² of the surface of the body part(s). In fact, due to the particular dosage of their ingredients, the cosmetic compositions according to the invention have a particularly advantageous consistency at normal application temperatures which makes it easy to apply these quantities to the skin without requiring additional intervention or special attention from the user. In particularly advantageous embodiments incorporating a stick, the regular and perfectly dosed application is even simpler, faster and more hygienic.

### EXAMPLES

The cosmetic compositions according to the invention are preferably in the form of a stick (heat cast cosmetic product) with a high clay content.

Their composition is preferably one of the following:

Examples of clay formula sticks

| INCI/MP name | % MP/% by weight of the formula |
|---|---|
| Water | 20-60 |
| Propylene glycol | 5-40 |
| Glycerin | 5-30 |
| Gellan gum | 0.01-5 |
| Sodium stearate | 3-10 |
| Sodium hydroxide | 0.01-2 |
| Clay, e.g. kaolin | 10-40 |
| Pigment + glycerin (50:50) | 0.5-10 |
| Fragrance | 0.1-2 |
| Preservative | 0.1-2 |
| Total | 100 |

Clay formula sticks - Example 1

| INCI/MP name | % MP/% by weight of the formula |
|---|---|
| Water | 47.02 |
| Propylene glycol | 10 |
| Glycerin | 10 |
| Gellan gum | 0.10 |
| Sodium stearate | 5.50 |
| Sodium hydroxide | 0.18 |
| Kaolin | 20 |
| Chromium oxide + glycerin | 1 |
| Titanium dioxide + glycerin | 5 |
| Fragrance | 0.30 |
| Phenoxyethanol + Ethylhexylglycerin | 0.90 |
| Total | 100 |

The formulation of Example 1 has good purifying and detoxifying properties and can be used especially for the purification of the skin of the face or scalp.

Although the invention has been described in detail for the purpose of illustration, it is understood that such details are solely for that purpose. The variations can be made therein by those skilled in the art without departing from the spirit of the scope of the invention which is defined by the following claims.

1. A cosmetic caro composition that can be heat cast and molded comprising from 10 to 40% by weight of one or more clays, 10 to 60% by weight of water, 5 to 70% by weight of one or more humectants, and 3 to 10% by weight of one or more viscosity agents.

2. The cosmetic composition according to claim 1, wherein the humectants are selected from the group consisting of urea, amino acids and their derivatives, organic acids and their salts, glycerin and all types of polyols, natural and synthetic derivatives of sugars and polysaccharides, hydrolysates of silk, keratin or soya, and their mixtures and combinations.

3. The cosmetic composition according to claim 2, wherein the humectants are propylene glycol, glycerin or a combination thereof.

4. The cosmetic composition according to claim 1, wherein the viscosity agent are fatty acids comprising a carbon chain having between 12 and 22 carbon atoms, neutralized by one or more organic or mineral bases, and their mixtures and combinations.

5. The cosmetic composition according to claim 1, further comprising: 0.5 to 10% of a dye, a mineral or organic pigment in solid form or in a slurry, an opacifying agent, pearlescent agents, flakes, exfoliating particles, and their mixtures or combinations.

6. The cosmetic composition according to claim 1, further comprising:
0.01 to 5% by weight of one or more suspending agents.

7. The cosmetic composition according to claim 1, further comprising 0.1 to 7% by weight of one or more cosmetics, emollients, emulsifiers, fragrances, preservatives and/or pH adjustment agents.

8. Cosmetic article comprising the cosmetic composition according to claim, wherein the cosmetic article is heat cast and molded.

9. The cosmetic article according to claim 8, wherein the cosmetic article is in the form of a stick.

10. A process for manufacturing a heat cast and molded cosmetic article comprising:
hot mixing 10 to 40% by weight of one or more clays, 10 to 60% by weight of water, 5 to 70% by weight of one or more humectants, and 3 to 10% by weight of one or more viscosity agents.

11. A method comprising:
applying the cosmetic composition according to claim 1 to a subject's body.

12. The method according to claim 11 further comprising:
removing the cosmetic composition from the subject's body by rinsing after said applying.

13. The method according to claim 11, wherein the cosmetic composition is applied in an amount of 1.5 to 10 mg/cm² of the surface the subject's body.

14. The method according to claim 11, wherein the cosmetic composition is in the form of a stick.

15. The method according to claim 11, wherein the cosmetic composition is applied to the skin, nails, hair, face, neck, torso, arms, hands, legs and/or feet of a subject's body.

16. The method according to claim 12, wherein said removing is carried out by rinsing with water 1 to 20 minutes after said applying.

17. The method according to claim 16, wherein the cosmetic composition is applied in an amount of 3 to 8 mg/cm² of the surface of the body.

18. The cosmetic composition according to claim 1, wherein the clay is selected from the group consisting of kaolin, bentonite, laponite, smectite, illites, chlorites, vermiculites, sepiolites, attapulgites and their derivatives, and their mixtures and combinations.

19. The cosmetic composition according to claim 3, wherein the humectant comprises from 1 to 40% by weight of propylene glycol and from 1 to 30% by weight of glycerin in claim 3.

20. The cosmetic composition according to claim 6, wherein the suspending agent is selected from the group consisting of xanthan gum, guar gum, locust bean gum, sclerotium gum, gellan gum, cellulose derivatives, synthetic polymers, and their mixtures and combinations.
